(19) United States
(12) Patent Application Publication
(10) Pub. No.: US 2022/0292865 A1
(43) Pub. Date: Sep. 15, 2022

(54) SPATIAL INFECTION RISK DETERMINATION SYSTEM, SPATIAL INFECTION RISK DETERMINATION METHOD, AND STORAGE MEDIUM

(71) Applicant: NEC Corporation, Tokyo (JP)

(72) Inventors: Tomoko NISHIO, Tokyo (JP); Akira KAMEI, Tokyo (JP); Kenichiro IDA, Tokyo (JP); Itsumi KATO, Tokyo (JP); Tomotaka SUZUKI, Tokyo (JP)

(73) Assignee: NEC Corporation, Tokyo (JP)

(21) Appl. No.: 17/672,214

(22) Filed: Feb. 15, 2022

(30) Foreign Application Priority Data
Mar. 9, 2021 (JP) 2021-036913

(51) Int. Cl. G06V 40/10; G06T 7/62; G16H 30/20; G16H 50/30

(52) U.S. Cl. CPC G06V 40/10; G06T 7/62; G16H 30/20; G16H 50/30

(57) ABSTRACT

A spatial infection risk determination system according to one aspect of the present disclosure includes: at least one memory configured to store instructions; and at least one processor configured to execute the instructions to: detect a person from a captured image in a space acquired by an imaging device; and determine an infection risk in the space based on a floor area of the space and an area of a circle whose center is the detected person, the area of the circle being based on a distance to prevent infection of an infectious disease.

This application is based upon and claims the benefit of priority from Japanese Patent Application No. 2021-036913, filed on Mar. 9, 2021, the disclosure of which is incorporated herein in its entirety by reference.

### TECHNICAL FIELD

The present disclosure relates to a spatial infection risk determination system, a spatial infection risk determination method, and a storage medium.

### BACKGROUND ART

A means that informs facility operators and prospective facility users of useful congestion information for ensuring the distance between people, which is necessary to prevent the spread of infectious diseases, is disclosed. (See, for example, Japanese Patent No. 6764214)

### SUMMARY

An object of the present disclosure is to provide a spatial infection risk determination system for determining an infection risk according to a state of a space.

A spatial infection risk determination system according to one aspect of the present disclosure includes: at least one memory configured to store instructions; and at least one processor configured to execute the instructions to: detect a person from a captured image in a space acquired by an imaging device; and determine an infection risk in the space based on a floor area of the space and an area of a circle whose center is the detected person, the area of the circle being based on a distance to prevent infection of an infectious disease.

A spatial infection risk determination method according to one aspect of the present disclosure includes: detecting a person in a space; and determining an infection risk in the space based on a floor area of the space and an area of a circle whose center is the detected person, the area of the circle being based on a distance to prevent infection of an infectious disease.

A non-transitory computer readable storage medium according to one aspect of the present disclosure stores a program for causing a processor of a computer to execute: detection processing of detecting a person in a space; and determination processing of determining an infection risk in the space based on a floor area of the space and an area of a circle whose center is the detected person, the area of the circle being based on a distance to prevent infection of an infectious disease.

### BRIEF DESCRIPTION OF THE DRAWINGS

Exemplary features and advantages of the present invention will become apparent from the following detailed description when taken with the accompanying drawings in which:

FIG. 1 is a diagram for describing an example of using a spatial infection risk determination system in the present example embodiment;

FIG. 2 is an example illustrating a configuration diagram of the spatial infection risk determination system in the present example embodiment;

FIG. 3 is a sequence diagram illustrating a flow of processing according to the present example embodiment;

FIG. 4 is a flowchart illustrating a flow of processing according to the present example embodiment;

FIG. 5 is an example of a processing screen in the present example embodiment;

FIG. 6 is a flowchart illustrating a flow of processing according to the present example embodiment;

FIG. 7 is an example of a processing screen in the present example embodiment;

FIG. 8 is a flowchart illustrating a flow of processing according to the present example embodiment;

FIG. 9 is an example of a processing screen in the present example embodiment;

FIG. 10 is an example of a processing screen in the present example embodiment;

FIG. 11 is a flowchart illustrating a flow of processing according to the present example embodiment;

FIG. 12 is a flowchart illustrating a flow of processing according to the present example embodiment;

FIG. 13 is an example illustrating a configuration diagram of the spatial infection risk determination system in the present example embodiment;

FIG. 14 is a sequence diagram illustrating a flow of processing according to the present example embodiment; and

FIG. 15 is an example of a hardware configuration of the spatial infection risk determination system in the present example embodiment.

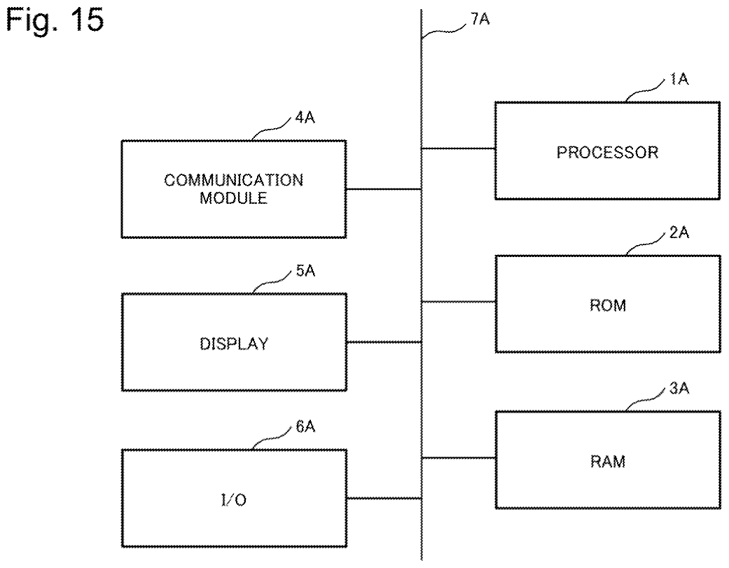

### EXAMPLE EMBODIMENT

First, background of example embodiments of the present disclosure will be described in order to facilitate understanding of embodiments of the present disclosure.

There are cases where facilities are desired to operate while giving consideration to preventing the spread of infectious diseases. For example, to prevent the spread of infectious diseases, it is important to secure distance (social distance) between people required to prevent the spread of infectious diseases. Until now, the social distance has often been set to a fixed distance on the basis of a flying distance of droplets emitted from the mouth of a person during conversation or coughing. However, when comparing adults and children, for example, it is assumed that adults will fly droplets farther due to their size or the like and has a higher risk of infecting people with infectious diseases (infection risk). Further, people with cold symptoms are considered to have a higher infection risk because they sneeze and cough more frequently than people without cold symptoms. Moreover, even when comparing spatial environments, if the humidity and temperature are low, virus is activated and the infection risk increases. As described above, the social distance required to prevent the spread of infection depends on various states within a space.

For example, in a case of determining a congestion status of a certain facility, the degree of congestion is sometimes determined by calculating the number of people per unit area of the space. If the value is higher than a predetermined value, a facility operator sometimes determines that the degree of congestion is high and determines that no more people are allowed to enter the space. However, in this case, whether people can enter the space is not determined in consideration of the infection risk that changes depending on various states in the space as described above.

According to the example embodiments of the present disclosure to be described below, it is possible to determine an infection risk in a space that changes depending on various states in the space. Hereinafter, an infection risk in a certain space is referred to as a spatial infection risk.

Hereinafter, example embodiments of the present disclosure will be described with reference to the drawings. Similar elements or corresponding elements may be designated by the same reference numerals in the drawings, and description thereof may be omitted or simplified.

### [Overview of Functions]

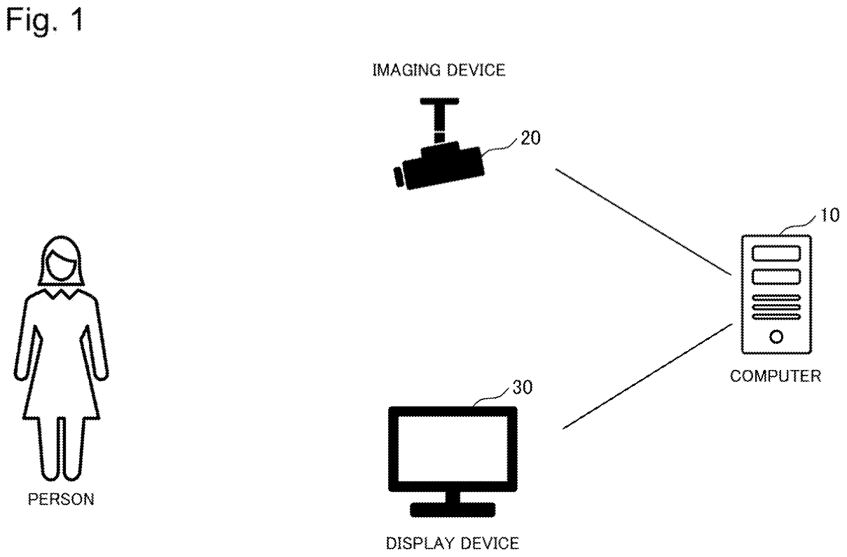

An outline of functions implemented by the present disclosure will be described with reference to FIG. 1. FIG. 1 illustrates a configuration of a spatial infection risk determination system according to the present example embodiment. As illustrated in FIG. 1, the spatial infection risk determination system according to the present example embodiment includes a computer 10, an imaging device 20, and a display device 30.

The computer 10 sets a social distance on the basis of an attribute and a state of a person detected by the imaging device 20 or an environmental condition of a space (room), and determines a spatial infection risk. The social distance is a distance between people required to prevent infection of an infectious disease, and is a distance to prevent the infection of an infectious disease.

The imaging device 20 is a picture acquisition unit such as a surveillance camera that is installed at a predetermined monitoring position or the like and captures a person in an imaging space. The imaging device 20 may be a camera with fixed orientation and installation location, a camera with changeable orientation such as a pan tilt zoom (PTZ) camera, or a movable camera mounted on a moving body such as a drone. Further, for example, the imaging device 20 may be a camera mounted on a wearable terminal such as a smartphone or a tablet. The imaging device 20 and the computer 10 are connected so as to be able to communicate with each other via an arbitrary network. The computer 10 and the imaging device 20 may be combined into one device.

The display device 30 displays a calculated social distance range in a display mode according to the risk of an infectious disease. The social distance range is a range (region) that defines the distance between people to prevent the infection of infectious diseases. For example, the display device may be a signage placed outside the space or a PC display. Further, the display device may be a display unit of a wearable terminal such as a smartphone or a tablet. The display device 30 and the computer 10 are connected so as to be able to communicate with each other via an arbitrary network. The computer 10, the imaging device 20, and the display device 30 may be combined into one device.

As described above, in the present example embodiment, when visualizing the social distance range necessary for infection prevention, the social distance range of a person can be appropriately calculated by setting the social distance according to the infection risk. In addition, a manager of the space can easily determine whether additional people can enter the space by the spatial infection risk determination system determining the spatial infection risk from the above-described social distance range.

### First Example Embodiment

A configuration of a spatial infection risk determination system 1 and a spatial infection risk determination method in the present example embodiment will be described with reference to FIG. 1. FIG. 1 is a diagram illustrating an overall configuration example of the spatial infection risk determination system 1 in the present example embodiment. The spatial infection risk determination system 1 includes a computer 10, an imaging device 20, and a display device 30. The computer 10 and the imaging device 20 can communicate with each other via wired or wireless communication units respectively provided therein. Further, similarly, the computer 10 and the display device 30 can communicate with each other via wired or wireless communication units respectively provided therein. The number of computers 10, imaging devices 20, and display devices 30 is at least one each, and a plurality of devices can be connected and installed at the same time.

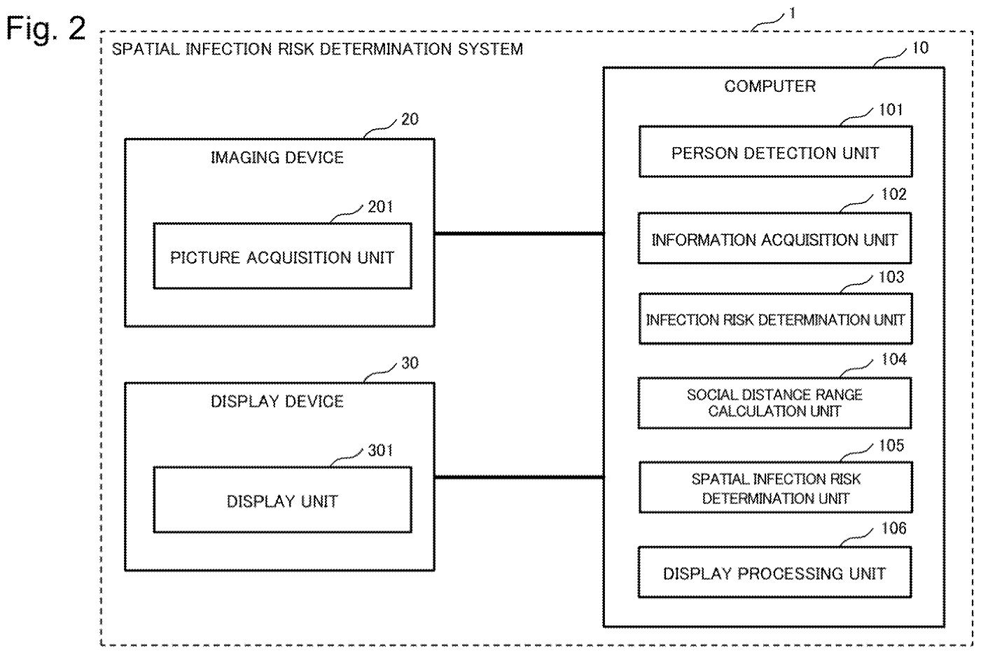

Next, functional configurations of the computer 10, the imaging device 20, and the display device 30 will be described with reference to FIG. 2. The computer 10 includes a person detection unit 101, an information acquisition unit 102, an infection risk determination unit 103, a social distance range calculation unit 104, a spatial infection risk determination unit 105, and a display processing unit 106. The imaging device 20 includes a picture acquisition unit 201, and the display device 30 includes a display unit 301. The person detection unit 101 serves as a detection means that detects a person from an image captured in a space acquired by the imaging device 20. The information acquisition unit 102, the infection risk determination unit 103, the social distance range calculation unit 104, and the spatial infection risk determination unit 105 serve as a determination means that determines an infection risk in a space according to a floor area of the space and an area of a circle whose center is the detected person and of the circle based on the distance to prevent infection of an infectious disease. The display processing unit 106 serves as a display means that displays the circle around the person on the display device 30.

The person detection unit 101 automatically identifies and detects the person from a picture obtained from the picture acquisition unit 201. This identification of the person is specifically performed by any of various known techniques or a combination of the techniques. For example, an object other than background is extracted from the captured image by background subtraction. This background subtraction is a known technique, and is a technique of extracting a moving object by taking a difference between image data of the captured image captured by the picture acquisition unit 201 and a background image of a capture target region acquired in advance. Another known technique example is a machine learning-type image analysis. In machine learning picture analysis, people in the picture image are automatically and efficiently identified by an image recognition technique using deep learning.

The information acquisition unit 102 acquires information of an attribute and a state of a person or an environmental condition of the space for setting a social distance. The attribute of a person is specifically a height or an age, and the state of a person is a state of physical condition such as whether the acquired person has a cold symptom or whether the acquired person has an underlying disorder. The environmental condition is a temperature, a humidity, an amount of ultraviolet rays, a carbon dioxide concentration, or the like of the space for determining the spatial infection risk.

The infection risk determination unit 103 determines the infection risk on the basis of the attribute and state of the person or the environmental condition of the space acquired by the information acquisition unit 102. For example, the attribute of the person is the height or age of the person. A tall person is determined to have a higher infection risk because the tall person is more likely to fly droplets farther than a short person, and the short person is determined to have a lower infection risk. The state of the person refers to the presence or absence of a cold symptom (sneezing, coughing, or fever) or the presence or absence of an underlying disorder of the person. A person with a cold symptom is determined to have a higher infection risk than a person without a cold symptom, and a person without a cold symptom is determined to have a lower infection risk.

Further, the environmental condition of the space refer to the temperature, humidity, amount of ultraviolet rays, or carbon dioxide concentration that can be observed within the space. Generally, it is determined that the infection risk is high under environmental conditions where the temperature is low, the humidity is low, and the amount of ultraviolet rays is low because a virus is easily activated. On the other hand, it is determined that the infection risk is low under environmental conditions where the temperature is high, the humidity is high, and the amount of ultraviolet rays is high. Further, it is determined that the infection risk is high in the space where the carbon dioxide concentration is high because it is considered that the space has not been ventilated for a long time. Here, the infection risk refers to a risk that a person who may be infected may infect an uninfected person, or a risk the an uninfected person becomes severe in the case where the uninfected person is infected.

The social distance range calculation unit 104 calculates a social distance range on the basis of the infection risk determined by the infection risk determination unit 103. For example, the social distance range is a circle with a radius R whose center is a person. Here, the radius R is the social distance, and is set by the social distance range calculation unit 104 on the basis of the infection risk determined by the infection risk determination unit 103. The circle may be superimposed on the feet of the person or around the waist. Further, the social distance range is not limited to a circle, and may be a three-dimensional semicircular sphere that imitates a fall range from the mouth to the ground, assuming a range in which droplets fly. The radius R is set on the basis of the high or low of the infection risk determined by the infection risk determination unit 103. In the present example embodiment, a method of setting the radius R according to the high or low of the infection risk has been described, but a fixed value may be set without considering the attribute and state of the person or the environmental condition in the space.

The spatial infection risk determination unit 105 determines the infection risk in a certain space on the basis of the floor area of the space to be determined and the social distance range of the person existing in the space. For example, the spatial infection risk is expressed as a sum of the social distance ranges of all the persons existing in the space with respect to the floor area of the space. The following equation 1 is an example of a mathematical equation for determining the spatial infection risk. A represents the spatial infection risk, R represents the radius of the social distance range, n represents the number of people existing in the space, and S represents the floor area in the space.

A=ΣRₙ²π/S [Math. 1]

When A exceeds 1, the space is determined to have a high spatial infection risk. In the above mathematical equation example, an overlap of the social distance ranges for each person is not considered when determining the spatial infection risk, but the spatial infection risk may be determined by focusing on the overlap of the social distance ranges. That is, it may be determined that the spatial infection risk is high in the case where the overlap of the social distance ranges exceeds a predetermined value with respect to the area of the space. Further, an alert is issued in the case where the spatial infection risk is high, and a person outside the space or the manager of the space is notified that the spatial infection risk is high.

The display processing unit 106 superimposes the social distance range calculated by the social distance range calculation unit 104 around the person captured in the picture acquired by the picture acquisition unit 201 and displays the superimposed picture on the display unit 301. As an example of the present example embodiment, a person inside or outside the space visually recognizes the infection risk by changing the size of the circle indicating the social distance range superimposed on the display device 30 according to the infection risk. Further, the infection risk can be easily recognized by changing the color of the social distance range or highlighting the social distance range with a thick line. Moreover, in a case of displaying the social distance range in blinking, a blinking interval may be changed. The above social distance range is a circle that represents a region in a predetermined distance range whose center is the body of a person located higher than the feet of the person, but may be a three-dimensional semicircular sphere that imitates a fall range from the mouth to the ground, assuming a range in which droplets fly.

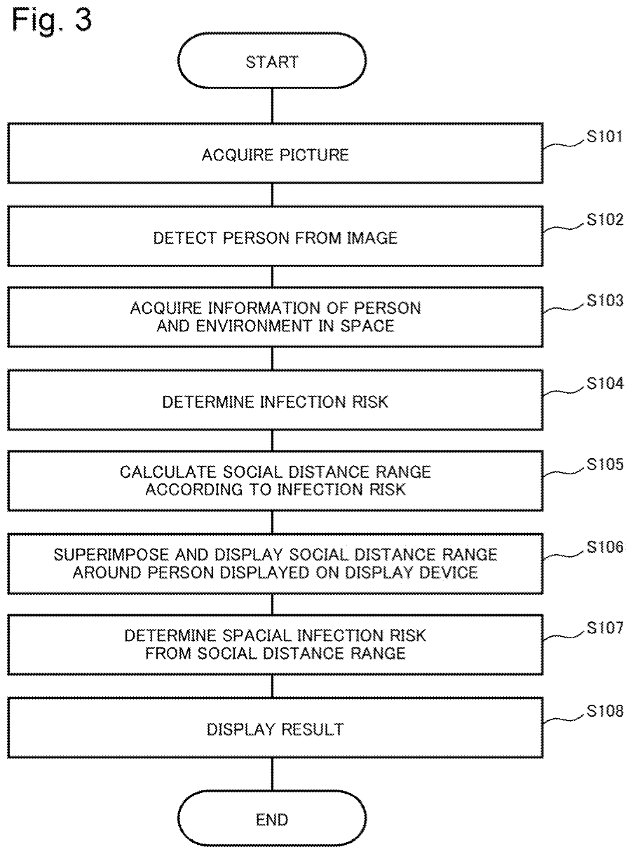

FIG. 3 is a sequence diagram illustrating a flow of processing of the present example embodiment from acquisition of a picture by the picture acquisition unit 201 to determination of the spatial infection risk and display on the display unit 301. The picture acquisition unit 201 acquires a picture in the space for determining the spatial infection risk (S101). The person detection unit 101 detects a person from the acquired picture (S102). Further, the information acquisition unit 102 acquires the information of the attribute and state of the person or the environmental condition of the space for setting the social distance (S103), and the infection risk determination unit 103 determines the high or low of the infection risk using the acquired information (S104). The social distance range calculation unit 104 calculates the social distance range on the basis of the determination of the infection risk determination unit 103 (S105). The display processing unit 106 superimposes and displays the social distance range calculated the social distance range calculated by S105 around the person displayed on the display device 30 (S106). The spatial infection risk determination unit 105 calculates a ratio of the sum of the social distance ranges of all the persons with respect to the floor area in the space as the spatial infection risk, and determines the spatial infection risk (S107). The display processing unit 106 displays a determination result (S108).

In the case where the spatial infection risk is 1 or higher, the spatial infection risk is determined to be high, and the alert is output to the manager of the space or on the display unit 301 outside the space. Further, a signage can be placed outside the space, and the display processing unit 106 can alert people who intend to enter the space by displaying a person on which the social distance range is superimposed or the presence or absence of the alert in a visually recognizable manner. Alternatively, there may be a method of posting alert information on a website in the space or sending a notification regarding the alert to a wearable terminal such as a smartphone. By the above technique, when a person enters the space, the infection risk that the person is infected with an infectious disease can be determined according to the state of the space, and the manager can easily determine whether to allow people to additionally enter the space.

### Second Example Embodiment

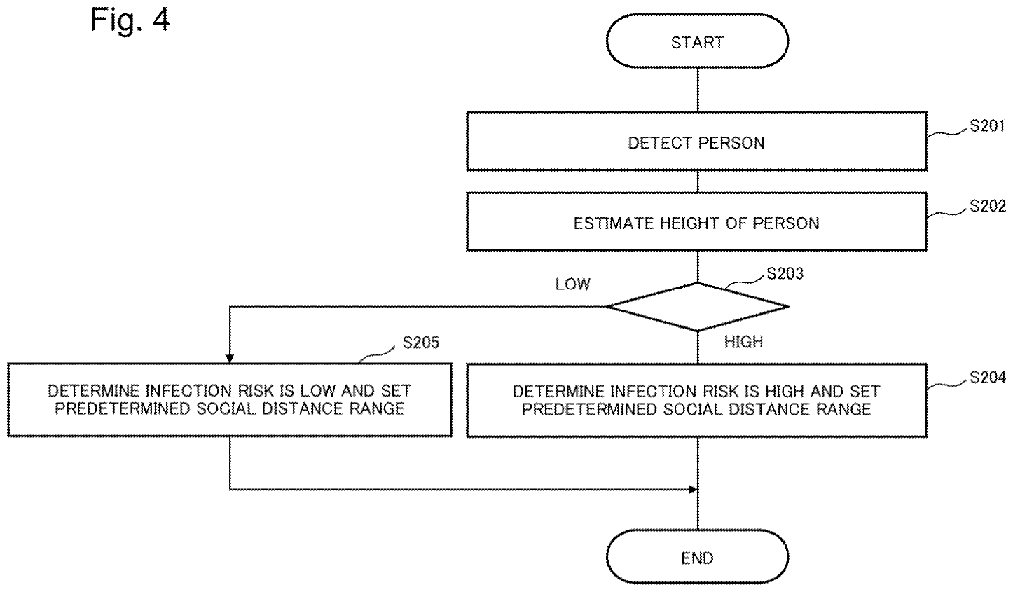

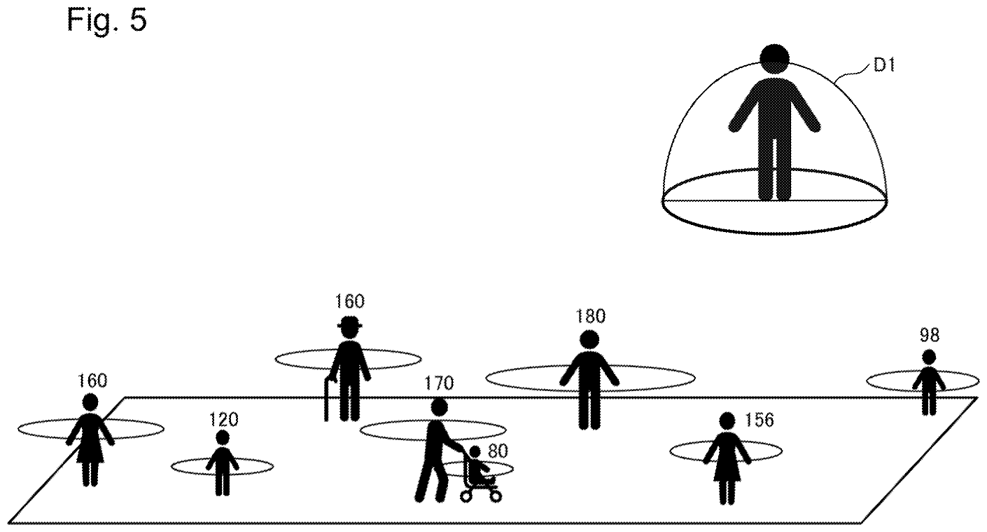

Next, another example applicable to the above-described first example embodiment will be described with reference to FIGS. 4 and 5. In the first example embodiment, the method in which the infection risk determination unit 103 determines the infection risk on the basis of the attribute and state of the person or the environmental condition of the space acquired by the information acquisition unit 102 has been described. In a second example embodiment, a method of determining an infection risk on the basis of a height, which is one of attributes of a person, will be described in more detail.

Generally, when a person sneezes, a shortest distance (flying distance) from the feet of the person to a position when droplets reach a floor is different between a case where the person sneezes while standing and a case where the person sneezes while sitting, even in the case where the same person sneezes. In other words, it is considered that sneezing droplets of a tall person take a long time to fall to the floor, and even if initial velocity of the droplets from the mouth is the same, the droplets fly farther than those of a short person. Therefore, the tall person more likely to infect those around him/her than the short person, and require a wider social distance range than the short person.

FIG. 4 is a flowchart illustrating a flow of processing according to the present example embodiment. A person detection unit 101 detects a person existing in a space (S201). Next, an information acquisition unit 102 estimates the height of the person detected by the person detection unit 101 (S202).

The height estimation method is performed by any of various known techniques or a combination of the techniques. For example, in a case where an imaging device 20 is a surveillance camera installed at an arbitrary position on a road or in a facility, an arbitrary object (a post, a vending machine, a signal, or the like) installed in a capture region is always present in a captured image. The actual height of this object may be measured in advance and input to the information acquisition unit 102. The information acquisition unit 102 estimates the height of each person on the basis of a relative positional relationship between each person detected from the captured image and the object, the height of the object in the image, and the actual height of the object. In addition, a feature amount of an appearance and the height of each of a plurality of persons may be registered in a database in advance.

The information acquisition unit 102 recognizes the detected person by comparing the feature amount of the appearance of the person detected in the captured image with the feature amount of the appearance registered in the database, and acquires the height of the recognized person from the database. After acquiring the height of each person, an infection risk determination unit 103 determines the high or low of an infection risk (S203). For example, a person with the height of equal to or shorter than 120 cm is determined to have a low infection risk, and a person with the height of higher than 120 cm is determined to have a high infection risk.

A social distance range calculation unit 104 calculates a social distance range on the basis of the determination of the infection risk (S204). For example, assuming that fall velocity of droplets is 30 cm/sec, the time for the droplets to reach the floor is 4 seconds for a person with the height of 120 cm, and 6 seconds for a person with the height of 180 cm. Assuming that the flying distance of the droplets from the feet of the person with the height of 180 cm is 6 m, initial velocity from the mouth of the droplets is 1 m/sec, and when this is applied to the person with the height of 120 cm, the flying distance of the droplets is calculated as 4 m.

In the flowchart of FIG. 4, description has been given, dividing the infection risk into two risks: high infection risk and low infection risk, but the example embodiment is not limited to the case and there may be an intermediate infection risk. For example, assuming that a person with the height of 150 cm has an intermediate infection risk, the social distance range is calculated as 5 m when the above calculation is applied to the person with the height of 150 cm. In other words, the social distance range is set to 4 m for the height of equal to or shorter than 120 cm, 5 m for the height from 120 cm to 150 cm, and 6 m for the height of equal to or higher than 150 cm. The above value of the social distance may be freely input to a system by a manager or may be randomly set to a program in advance. Further, determination of the infection risk is not limited to the above method and may be determined in multiple stages.

FIG. 5 is an example of a processing screen in the present example embodiment. An estimated height is displayed above the head of a person existing in the space. Further, a circle indicating the social distance range based on the infection risk is superimposed around the person. In the present example, a display example of superimposing the height is illustrated but the height may not be displayed. Further, the circle representing the above social distance range is a circle that represents a region in a predetermined distance range whose center is the body of a person located higher than the feet of the person, but may be a three-dimensional semicircular sphere (D1) that imitates a fall range from the mouth to the ground, assuming a range in which droplets fly. By the above method, it is possible to set a more appropriate social distance range by considering the height rather than setting the same social distance to persons in the space.

### Third Example Embodiment

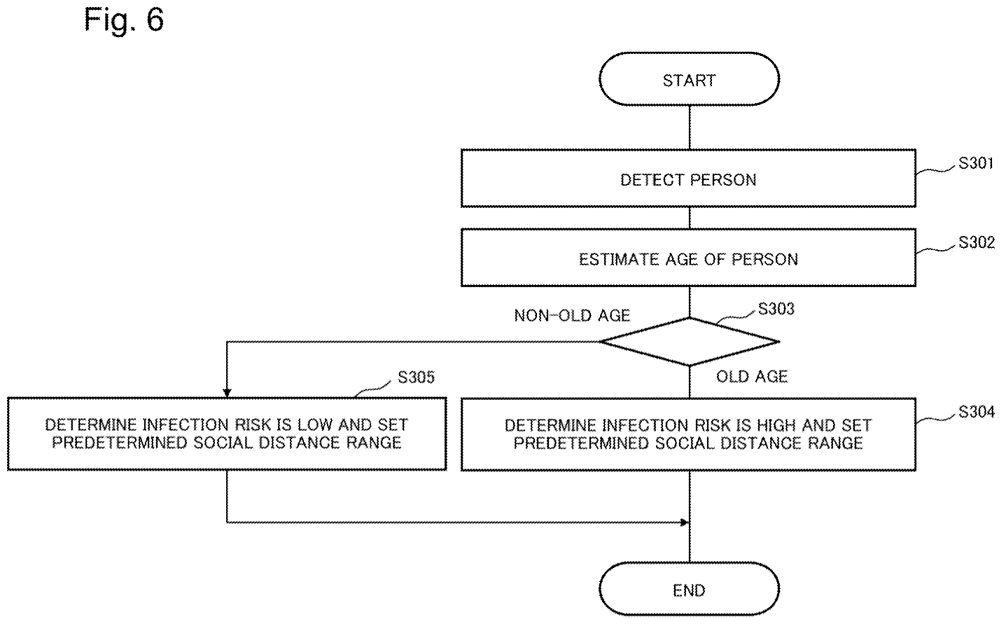

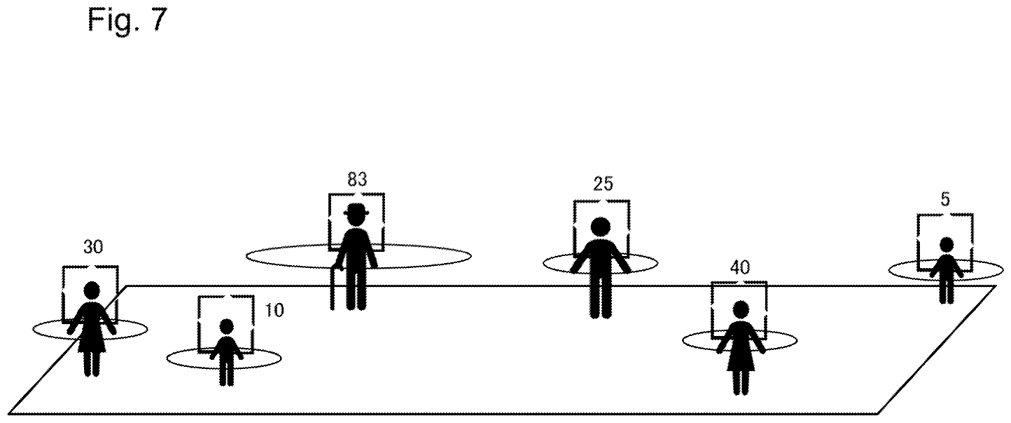

Next, another example applicable to the above-described first example embodiment will be described with reference to FIGS. 6 and 7. In the first example embodiment, the method in which the infection risk determination unit 103 determines the infection risk on the basis of the attribute and state of the person or the environmental condition of the space acquired by the information acquisition unit 102 has been described. In a third example embodiment, a method of determining an infection risk on the basis of an age, which is one of attributes of a person, will be described in more detail.

In general, older people who have an infectious disease are at higher risk of becoming more severe than younger people. For example, in the case of COVID-19, assuming that a severity rate in 30s is 1, the severity rate is about 25 times for 60s, about 50 times for 70s, about 70 times for 80s, and about 80 times for 90s or above. Here, severity is a case where treatment in an intensive care unit, use of a ventilator, or use of an extracorporeal membrane oxygenation is applicable.

FIG. 6 is a flowchart illustrating a flow of processing according to the present example embodiment. A person detection unit 101 detects a person existing in a space (S301). Next, an information acquisition unit 102 estimates the age of the person detected by the person detection unit 101 (S302).

The age estimation method is performed by any of various known techniques or a combination of the techniques. For example, a technique for learning feature amounts related to shape, wrinkles, spots, sagging, and color of the person's face, hair, and the like and estimating the age is disclosed.

After the age is estimated by the information acquisition unit 102, an infection risk determination unit 103 determines that, for example, a person estimated to be 65-year old or above has an old age and has a high infection risk. In addition, a person estimated to be under 65-year old is determined to have a young age and has a low infection risk. A social distance range calculation unit 104 sets a social distance to 5 m when the infection risk is determined to be high, and sets the social distance to 3 m when the infection risk is determined to be low.

Further, in the flowchart of FIG. 6, description has been given, dividing the infection risk into two risks: high infection risk and low infection risk, but the example embodiment is not limited to the case and there may be an intermediate infection risk. For example, a person estimated to be 65-year old or above is determined to have a high infection risk, a person estimated to be from 40-year old to under 65-year old is determined to have an intermediate infection risk, and a person estimated to be under 40-year old is determined to have a low infection risk. In this case, the social distance range calculation unit 104 may set the social distance to 5 m when the infection risk is determined to be high, set the social distance to 4 m when the infection risk is determined to be intermediate, and set the social distance to 3 m when the infection risk is determined to be low. The above value of the social distance may be freely input to a system by a manager or may be randomly set to a program in advance. Further, determination of the infection risk is not limited to the above method and may be determined in multiple stages.

FIG. 7 is an example of a processing screen in the present example embodiment. An estimated age is displayed above the head of a person existing in the space. Further, a circle indicating the social distance range based on the infection risk is superimposed around the person. In the present example, a display example of superimposing the age is illustrated but the age may not be displayed. By the above method, it is possible to set a more appropriate social distance range by considering the age rather than setting the same social distance to persons in the space.

### Fourth Example Embodiment

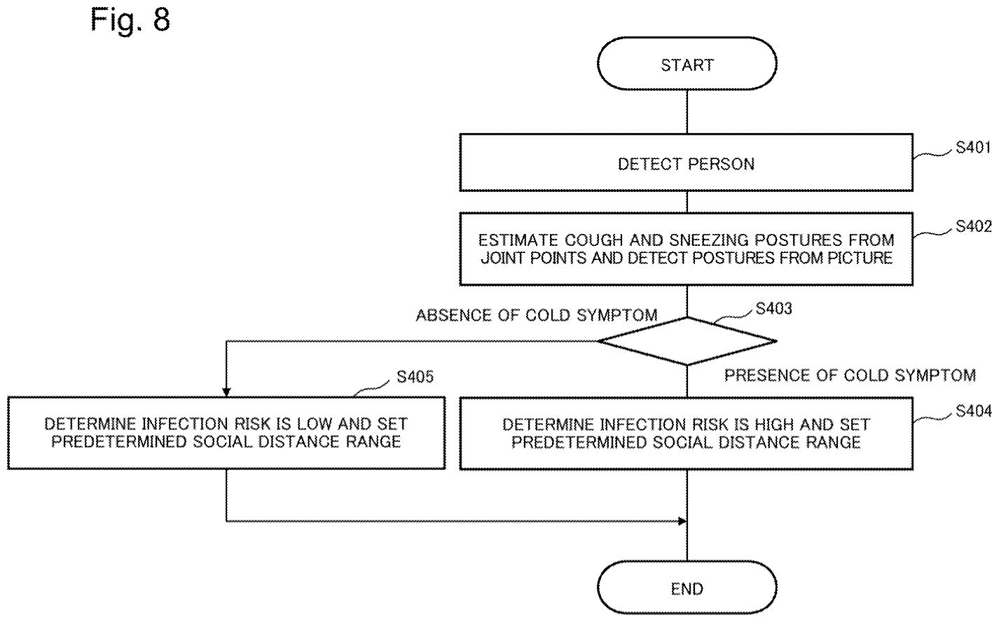

Next, another example applicable to the above-described first example embodiment will be described with reference to FIGS. 8 to 10. In the first example embodiment, the method in which the infection risk determination unit 103 determines the infection risk on the basis of the attribute and state of the person or the environmental condition of the space acquired by the information acquisition unit 102 has been described. In a fourth example embodiment, a method of determining an infection risk on the basis of presence or absence of a disease such as a cold symptom, which is one of states of a person, will be described in more detail.

Generally, a guideline for flying distance of droplets is 1 m for conversation, 3 m for coughing, and 5 m for sneezing. Further, in the case of an influenza patient, there is data that the amount of virus released by one cough is one hundred thousand, and is two million by one sneeze. In other words, in the case where a person has a cold symptom, there is an increased risk of infecting the people around him.

FIG. 8 is a flowchart illustrating a flow of processing according to the present example embodiment. A person detection unit 101 detects a person existing in a space (S401). Next, an information acquisition unit 102 acquires the presence or absence of a cold symptom detected by the person detection unit 101 (S402).

The acquisition of the presence or absence of a cold symptom is performed by any of various known techniques or a combination of the techniques. For example, a joint estimation technique (skeleton estimation technique) such as Open Pose using machine learning is used. The cough and sneeze are detected from a shape and reflexive movement of the entire joints of the person detected by the person detection unit 101 by the joint estimation technique.

After the acquisition of the presence or absence of a cold symptom by the information acquisition unit 102, the infection risk determination unit 103 determines that the infection risk is high in the presence of a cold symptom, and determines that the infection risk is low in the absence of a cold symptom (S403). A social distance range calculation unit 104 sets a predetermined value such as a social distance to 5 m when the infection risk is determined to be high, and the social distance to 2 m when the infection risk is determined to be low (S404).

Further, in the flowchart of FIG. 8, description has been given, dividing the infection risk into two risks: high infection risk and low infection risk, but the example embodiment is not limited to the case and there may be an intermediate infection risk. For example, the infection risk is determined to be high in the case of detecting a sneeze, the infection risk is determined to be intermediate in the case of detecting a cough, and the infection risk is determined to be low in the absence of a cold symptom. In this case, the social distance range calculation unit 104 may set the social distance to 5 m when the infection risk is determined to be high, set the social distance to 3 m when the infection risk is determined to be intermediate, and set the social distance to 2 m when the infection risk is determined to be low. The above value of the social distance may be freely input to a system by a manager or may be randomly set to a program in advance.

Further, sneezing or coughing only once may not be associated with infection. Therefore, in the case where a next cold symptom has not been detected for a certain period of time since detection of a cold symptom, the infection risk may be determined to be lowered by one level or may be determined to be low.

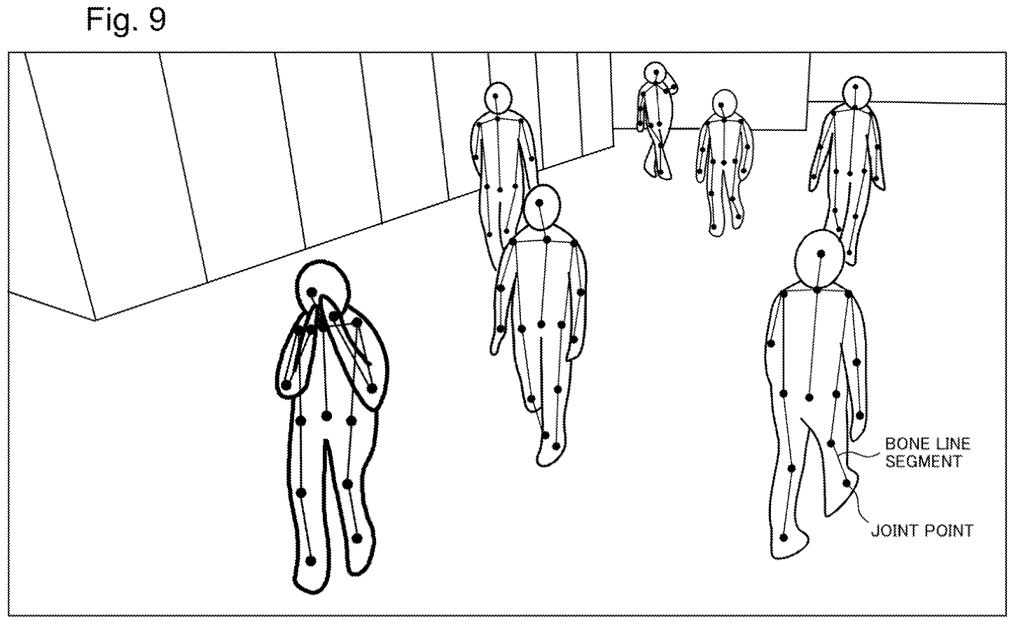

FIG. 9 is an example of a processing screen in the present example embodiment. The information acquisition unit 102 detects joint points presumed to be joints of a person on the basis of features of each part of the person recognized in an image, using the joint estimation technique, and further extracts a bone line segment connecting the detected joint points. Note that the detected joint points and bone line segments are the joint points and bone line segments estimated by the joint estimation technique, and may not match the joints and bones of an actual person. It can be said that the information acquisition unit 102 detects a skeletal structure including the joint points and bone line segments from the feet to the head of the person. The information acquisition unit 102 recognizes persons in a plurality of time-series images (frames), and detects joint points and bone line segments of all the persons recognized in each image. According to the above technique, the information acquisition unit 102 detects coughing or sneezing from the shape and reflexive movement of the entire joints of the person, such as the person putting his/her hand on the mouth.

In the present example, sneezing and coughing are described as examples for detecting the cold symptom but fever may be detected in addition to the above. An infrared camera is further installed as an imaging device 20, and the person detection unit 101 detects a person existing in the space. Next, the information acquisition unit 102 detects the body temperature of the person existing in the space, using the infrared camera. The infection risk determination unit 103 determines that the infection risk is high in the case where the body temperature detected by the information acquisition unit 102 is equal to or higher than 37.5° C., and the infection risk is low in the case where the body temperature is less than 37.5° C. The social distance range calculation unit 104 sets a predetermined value such as the social distance to 5 m when the infection risk is determined to be high, and the social distance to 2 m when the infection risk is determined to be low.

Further, a predetermined condition may be set by combining the presence or absence of a cold symptom and the detection of a fever, and the high or low of the infection risk may be determined. The above value of the social distance may be freely input to a system by a manager or may be randomly set to a program in advance.

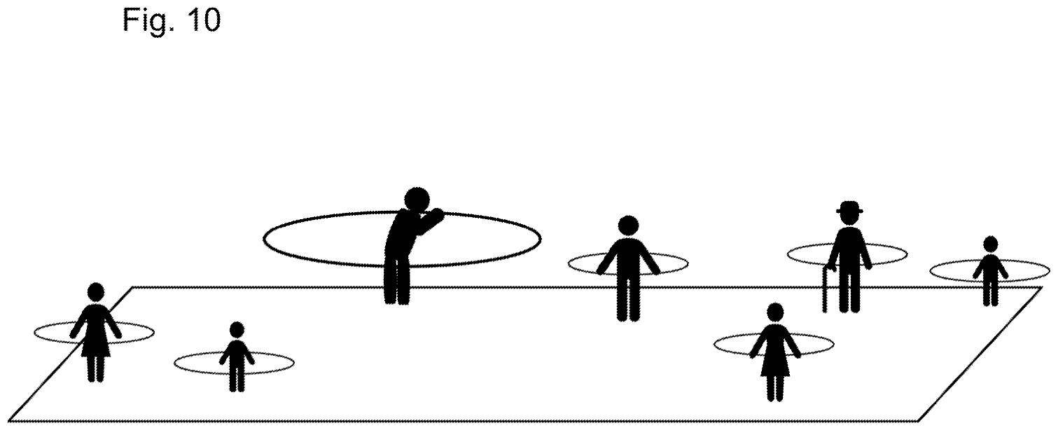

FIG. 10 is an example of a processing screen in the present example embodiment. A circle indicating a social distance range based on the infection risk is superimposed around the person. A display processing unit 106 may display the circle in an easy-to-understand manner for a manager by changing the color of the circle indicating the social distance range of the person having the infection risk, displaying the line of the circle thickly, or highlighting the circle. By the above method, it is possible to set a more appropriate social distance range by considering the cold symptom rather than setting the same social distance to persons in the space.

### Fifth Example Embodiment

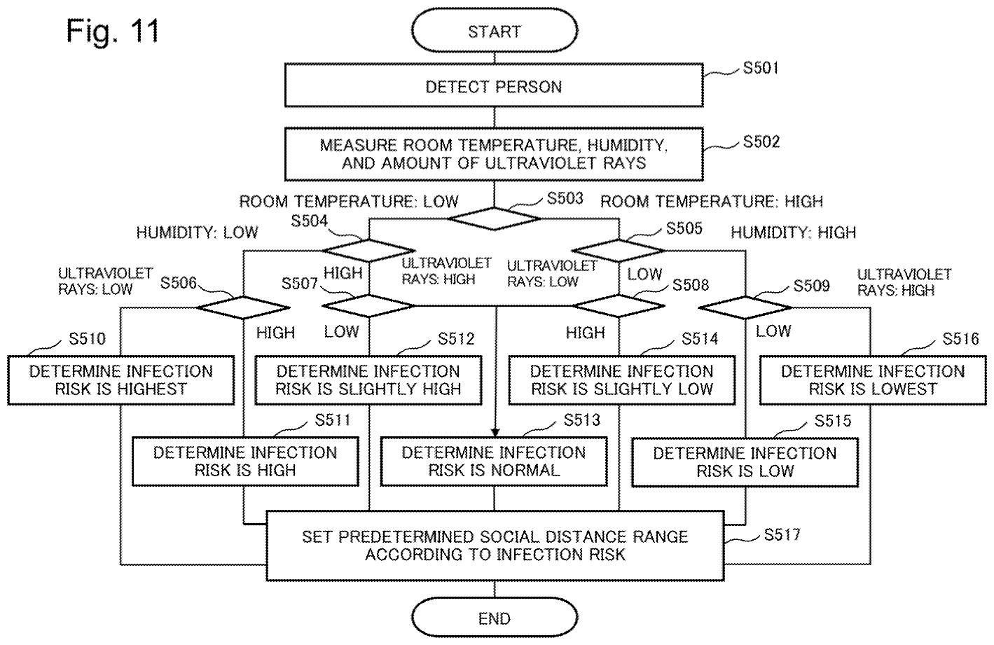

Next, another example applicable to the above-described first example embodiment will be described with reference to FIG. 11. In the first example embodiment, the method in which the infection risk determination unit 103 determines the infection risk on the basis of the attribute and state of the person or the environmental condition of the space acquired by the information acquisition unit 102 has been described. In a fifth example embodiment, a method of determining an infection risk on the basis of an environmental condition of a space will be described in more detail.

Common cold and influenza infectious diseases are inactivated under an environment with a high room temperature, a high humidity, and a high amount of ultraviolet rays, and are conversely activated under an environment with a low temperature, a low humidity, and a low amount of ultraviolet rays.

FIG. 11 is a flowchart illustrating a flow of processing according to the present example embodiment. A person detection unit 101 detects a person existing in a space (S501). Next, an information acquisition unit 102 acquires the room temperature, the humidity, and the amount of ultraviolet rays from a thermometer, a hygrometer, and an ultraviolet measuring device installed in a room (S502). In the present example embodiment, the thermometer, the hygrometer, and the ultraviolet measuring device are connected to a computer 10 by a wired or wireless manner.

After the acquisition of the room temperature, the humidity, and the amount of ultraviolet rays by the information acquisition unit 102, an infection risk determination unit 103 determines the infection risk on the basis of the high or low of the room temperature, the humidity, and the amount of ultraviolet rays. For example, the information acquisition unit 102 determines that the infection risk is the lowest in the case where the room temperature is high (S503), the humidity is high (S505), and the amount of ultraviolet rays is high (S509) (S516). Further, the information acquisition unit 102 determines that the infection risk is low in the case where the room temperature is high (S503), the humidity is high (S505), and the amount of ultraviolet rays is low (S509) (S515). Further, the information acquisition unit 102 determines that the infection risk is slightly low in the case where the room temperature is high (S503), the humidity is low (S505), and the amount of ultraviolet rays is high (S508) (S514). Further, the information acquisition unit 102 determines that the infection risk is normal in the case where the room temperature is high (S503), the humidity is low (S505), and the amount of ultraviolet rays is low (S508) (S513). Similarly, the information acquisition unit 102 determines that the infection risk is the highest in the case where the room temperature is low (S503), the humidity is low (S504), and the amount of ultraviolet rays is low (S506) (S510). Further, the information acquisition unit 102 determines that the infection risk is high in the case where the room temperature is low (S503), the humidity is low (S504), and the amount of ultraviolet rays is high (S506) (S511). Further, the information acquisition unit 102 determines that the infection risk is slightly high in the case where the room temperature is low (S503), the humidity is high (S504), and the amount of ultraviolet rays is low (S507) (S512). Further, the information acquisition unit 102 determines that the infection risk is normal in the case where the room temperature is low (S503), the humidity is high (S504), and the amount of ultraviolet rays is high (S507) (S513).

A social distance range calculation unit 104 sets a social distance to 4.5 m when the infection risk is determined to be the highest, sets the social distance to 4 m when the infection risk is determined to be high, and sets the social distance to 3.5 m when the infection risk is determined to be slightly high. Further, the social distance is set to 3 m when the infection risk is determined to be normal. Similarly, the social distance range calculation unit 104 sets a predetermined value such as the social distance to 2.5 m when the infection risk is determined to be slightly low, sets the social distance to 2 m when the infection risk is determined to be low, and sets the social distance to 1.5 m when the infection risk is determined to be the lowest (S517). The above value of the social distance may be freely input to a system by a manager or may be randomly set to a program in advance.

Further, in the flowchart of FIG. 11, the determination has been made, dividing the infection risk into seven stages, but the example embodiment is not limited to the case, and the number of stages may be reduced. For example, the number of determination stages may be limited to five by regarding the slightly low infection risk and the low infection risk as the same infection risk, and regarding the slightly high infection risk and the high infection risk as the same infection risk.

In FIG. 11, the determination has been made in the order of the room temperature, the humidity, and the amount of ultraviolet rays, but the order may be the amount of ultraviolet rays, the humidity, and the room temperature, and is not limited to the above case. In the present example, the three conditions (the room temperature, the humidity, and the amount of ultraviolet rays) are used as elements of the environmental conditions but the determination may be made using only one or two elements among the three elements.

There is also a method using an amount of carbon dioxide as another element. It is considered that the space is not ventilated in the case where the amount of carbon dioxide emitted by people in the space is large. The infection risk is determined to be high in the case where the amount of carbon dioxide exceeds a predetermined value, and the infection risk is determined to be low in the case where the amount of carbon dioxide does not exceed the predetermined value. A social distance range is set on the basis of a determination result. In the present example, the social distance is not set for each individual, but the social distance of all the persons existing in the space is the same fixed value. By the above method, it is possible to set a more appropriate social distance range by considering the environmental conditions in the space rather than setting the same social distance to persons in the space.

### Sixth Example Embodiment

Next, another example applicable to the above-described first example embodiment will be described. For example, an evacuation center may be used in the event of an emergency such as a disaster. There are only a limited number of places where people can evacuate in the emergency, and it may be difficult to use the evacuation center while taking into account an infection risk. In the present example, a method for determining a spatial infection risk in an evacuation center used in an emergency will be described in detail.

In the present example embodiment, an imaging device 21 (not illustrated) is installed at an entrance of the evacuation center in addition to an imaging device 20 that captures an inside of a space. Further, as additional functions of the computer 10 used in the first example embodiment, there are a face collation unit 107 (not illustrated) and a personal information registration unit 108 (not illustrated). The imaging device 21 acquires a face image of a person before entering the evacuation center. The imaging device 20 and the computer 10 can communicate with each other via a wired or wireless communication unit. The face image acquired by the imaging device 21 is transmitted to the personal information registration unit 108 of the computer 10. The personal information registration unit 108 inputs and registers a name, the presence or absence of an underlying disorder, the presence or absence of cold symptoms, and personal information such as an address and a telephone number, of the person entering the evacuation center, in addition to the face image. Here, the underlying disorder refers to chronic heart disease, respiratory disease, kidney disease, diabetes being treated with insulin, or the like, neurological disease and neuromuscular disease associated with immune abnormality, chromosomal abnormality, or the like. There is concern that a person with the above-described underlying disorder may become severe if he/she contracts an infectious disease such as COVID-19.

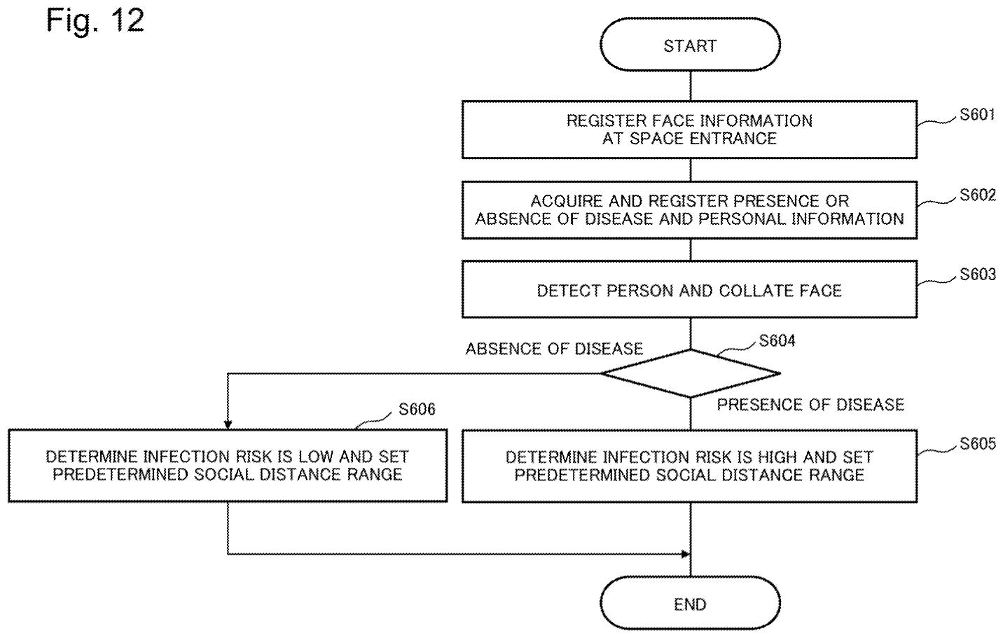

FIG. 12 is a flowchart illustrating a flow of processing according to the present example embodiment. The imaging device 21 installed at the entrance of the evacuation center acquires the face image of the person entering the evacuation center and transmits the face image to the personal information registration unit 108 (S601). The personal information registration unit 108, which has received the face image, acquires and registers the personal information of the person entering the evacuation center and information of the presence or absence of an underlying disorder and the presence or absence of cold symptoms, using an input unit such as a keyboard and a mouse of the computer 10 (S602). A person detection unit 101 detects a person existing in the space, and the face collation unit 107 collates face information of the detected person with the face image registered in the personal information registration unit 108 (S603).

An infection risk determination unit 103 refers to the personal information registration unit 108, and determines that the infection risk is high in the case where the collated person has an underlying disorder or a cold symptom, and determines that the infection risk is low in the case where there is no underlying disorder or cold symptom (S604). A social distance range calculation unit 104 sets a predetermined value such as a social distance to 5 m when the infection risk is determined to be high, and the social distance to 2 m when the infection risk is determined to be low (S605). The above value of the social distance may be freely input to a system by a manager or may be randomly set to a program in advance. The spatial infection risk described in the first example embodiment is determined on the basis of the social distance set for each individual.

By the above processing, it becomes easy to determine whether to allow people to additionally enter the evacuation center while considering the spatial infection risk. Further, by displaying the spatial infection risk on a signage or the like, a person who has once registered the personal information can individually determine whether to enter a room without relying on the manager. Further, as a secondary effect, since the face information is registered in the present example, if a family member or the like has a photo of the person in the evacuation center, whether the person is in the evacuation center can be confirmed using a face recognition technique. Alternatively, the person may be searched for using the name registered in the personal information registration unit 108. Moreover, if information of various evacuation centers can be integrated instead of just one evacuation center, who is evacuating to which evacuation center can be grasped.

### Seventh Example Embodiment

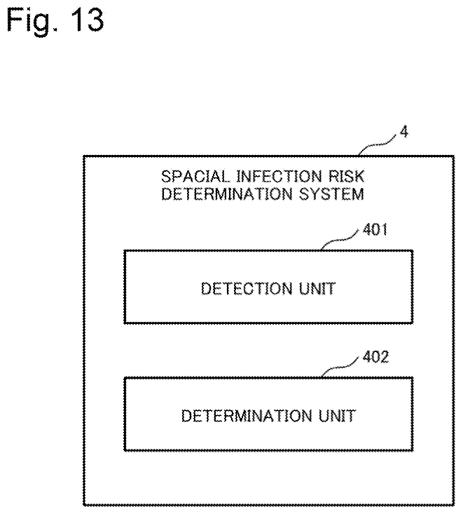

A minimum configuration of a spatial infection risk determination system 4 in the present disclosure will be described with reference to FIG. 13. FIG. 13 is an example illustrating an overall configuration example of the spatial infection risk determination system 4 in the present example embodiment. The spatial infection risk determination system 4 includes a detection unit 401 and a determination unit 402.

The detection unit 401 detects a person in a space for determining a spatial infection risk, using an imaging device 20. The determination unit 402 determines the spatial infection risk on the basis of a floor area of the space and an area of a circle based on a distance to prevent an infectious disease of all of persons existing in the space.

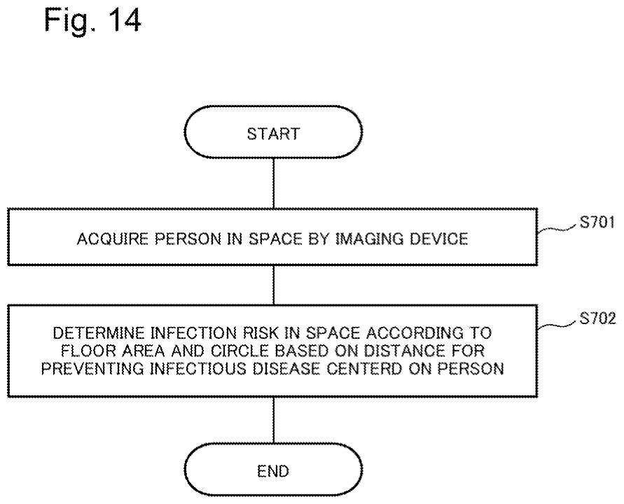

Next, a flow of processing related to the minimum configuration of the spatial infection risk determination system 4 will be described with reference to FIG. 14. The detection unit 401 detects a person in the space for determining the spatial infection risk, using the imaging device 20 (S701). The determination unit 402 determines the spatial infection risk according to the floor area of the space and the area of a circle based on a distance to prevent an infectious disease of all of persons existing in the space (S702).

From the above processing, a space manager can determine the spatial infection risk based on the grounds using a computer 10.

### Hardware Configuration Example

Next, an example of a hardware configuration that implements the computer 10, the imaging device 20, the display device 30, and the spatial infection risk determination system (1 or 4) in each of the above-described example embodiments will be described. The functional units of the computer 10, the imaging device 20, the display device 30, and the spatial infection risk determination system (1 or 4) are implemented by an arbitrary combination of hardware and software mainly including at least one central processing unit (CPU) of any computer, at least one memory, a program loaded in the memory, at least one storage unit such as a hard disk for storing the program, and a network connection interface. It is understood by those skilled in the art that there are various modifications for this implementation method and device. The storage unit can store a program stored before the device is shipped as well as a program downloaded from a storage medium such as an optical disk, a magneto-optical disk, or a semiconductor flash memory, or a server on the Internet.

A processor (1A) is, for example, an arithmetic processing unit such as a CPU, a graphics processing unit (GPU), or a microprocessor, and executes various programs or controls each part. That is, the processor (1A) reads the program from a ROM (2A) and executes the program using a RAM (3A) as a work area. In the above example embodiments, an execution program is stored in the ROM (2A).

The ROM (2A) stores the execution program for causing the processor (1A) to execute detection processing of detecting a person in a space, and determination processing of determining the infection risk in the space according to the floor area of the space and the area of the circle based on the distance to prevent infection of an infectious disease, and data related to the social distance range. The RAM (3A) temporarily stores the program and data as a work area.

A communication module (4A) implements a function in which the computer 10 communicates with the imaging device 20 and the display device 30. Further, in the case where a plurality of computers 10 is installed, the communication module implements the function to communicate with one another.

A display (5A) functions as a display unit, and has functions to input a request from a user with a touch panel, mouse, or the like, display a response from the spatial infection risk determination system (1 or 4), and display an image on which the social distance range is superimposed and a spatial infection risk result.

An I/O (6A) includes an interface for acquiring information from an input device, an external device, an external storage unit, an external sensor, a camera, or the like, and an interface for outputting information to an output device, an external device, an external storage unit, or the like. The input device is, for example, a touch panel, a keyboard, a mouse, a microphone, a camera, or the like. The output device is, for example, a display, a speaker, a printer, a lamp, or the like. The external sensor is, for example, a thermometer, a hygrometer, an ultraviolet ray amount measuring device, an infrared sensor, a carbon dioxide concentration meter, or the like.

The congestion information notification system described in JP 6764214 B discloses a technique of superimposing and displaying a circular line indicating a predetermined range from a person on a processed image. However, with the technique described in JP 6764214 B, when a person enters a space, the infection risk of the person to be infected with an infectious disease cannot be determined according to a state of the space.

According to the present disclosure, a spatial infection risk determination system, a spatial infection risk determination method, and a program according to a state of a certain space can be provided by setting the distance required for preventing infection of an infectious disease on the basis of the state of the space.

The configurations of the above-described example embodiments may be combined or some components may be replaced. Further, the configuration of the present disclosure is not limited only to the above-described example embodiments, and various modifications may be made without departing from the gist of the present disclosure.

1. A spatial infection risk determination system comprising:
at least one memory configured to store instructions; and
at least one processor configured to execute the instructions to:
detect a person from a captured image in a space acquired by an imaging device; and
determine an infection risk in the space based on a floor area of the space and an area of a circle whose center is the detected person, the area of the circle being based on a distance to prevent infection of an infectious disease.

2. The spatial infection risk determination system according to claim 1, wherein
the at least one processor is further configured to
display the circle around the person on a display device.

3. The spatial infection risk determination system according to claim 1, wherein
the circle based on the distance is the circle centered on the person, and
a radius of the circle is a predetermined fixed value.

4. The spatial infection risk determination system according to claim 1, wherein
a radius of the circle is set based on a height of the person.

5. The spatial infection risk determination system according to claim 1, wherein
a radius of the circle is set based on at least one of a temperature, a humidity, or an amount of ultraviolet rays in the space.

6. The spatial infection risk determination system according to claim 1, wherein
a radius of the circle is set based on presence or absence of a disease of the person.

7. The spatial infection risk determination system according to claim 1, wherein
a radius of the circle is set based on an age of the person.

8. The spatial infection risk determination system according to claim 1, wherein
a radius of the circle is set based on presence or absence of a fever of the person.

9. A spatial infection risk determination method comprising:
detecting a person in a space; and
determining an infection risk in the space based on a floor area of the space and an area of a circle whose center is the detected person, the area of the circle being based on a distance to prevent infection of an infectious disease.

10. The spatial infection risk determination method according to claim 9, further comprising
displaying the circle around the person on a display device.

11. The spatial infection risk determination method according to claim 9, wherein
the circle based on the distance is the circle centered on the person, and
a radius of the circle is a predetermined fixed value.

12. The spatial infection risk determination method according to claim 9, wherein
a radius of the circle is set based on a height of the person.

13. The spatial infection risk determination method according to claim 9, wherein
a radius of the circle is set based on at least one of a temperature, a humidity, or an amount of ultraviolet rays in the space.

14. The spatial infection risk determination method according to claim 9, wherein
a radius of the circle is set based on presence or absence of a disease of the person.

15. The spatial infection risk determination method according to claim 9, wherein
a radius of the circle is set based on an age of the person.

16. The spatial infection risk determination method according to claim 9, wherein
a radius of the circle is set based on presence or absence of a fever of the person.

17. A non-transitory computer readable storage medium storing a program for causing a processor of a computer to execute:
detection processing of detecting a person in a space; and
determination processing of determining an infection risk in the space based on a floor area of the space and an area of a circle whose center is the detected person, the area of the circle being based on a distance to prevent infection of an infectious disease.

18. The storage medium according to claim 17, wherein
the program causes the processor to execute
display processing of displaying the circle around the person on a display device.

19. The storage medium according to claim 17, wherein
the circle based on the distance is the circle centered on the person, and
a radius of the circle is a predetermined fixed value.

20. The storage medium according to claim 17, wherein
a radius of the circle is set based on a height of the person.
